(19) United States
(12) Patent Application Publication
(10) Pub. No.: US 2005/0060036 A1
(43) Pub. Date: Mar. 17, 2005

(54) Spinal column implant

(76) Inventors: Robert Schultz, Tuttlingen (DE); Jens Beger, Tuttlingen (DE); Stephan Lindner, Tuttlingen (DE); Jorg Schumacher, Tuttlingen (DE); Ulrich Kramer, Tuttlingen (DE); Uwe Bader, Tuttlingen (DE); Stefan Wallstein, Tuttlingen (DE)

Correspondence Address: RATNERPRESTIA, P O BOX 980, VALLEY FORGE, PA, 19482-0980, US

(21) Appl. No.: 10/885,370

(22) Filed: Jul. 6, 2004

(30) Foreign Application Priority Data
Jul. 24, 2003 (DE) DE 103 33 659.1

(51) Int. Cl. A61F 2/44

(52) U.S. Cl. 623/17.15; 623/17.12

(57) ABSTRACT

A spinal column implant is provided for contact with a vertebral body. The implant includes at least one contact surface including a contact plane having a cross-sectional area in contact with the vertebral body. The shape and arrangement of the at least one contact surface is adjustable such that the cross-sectional area of the contact plane is larger in a contact position than in an insertion position.

This application is related to and claims the benefit of German Utility Model No. 203 11 400.0 entitled Spinal Column Implant issued on Oct. 2, 2003, and German Patent Application No. 103 33 659.1 filed Jul. 24, 2003.

### FIELD OF THE INVENTION

The present invention pertains to intervertebral and vertebral implants, with which the original height of the intervertebral disk or vertebral body can be restored in case of, e.g., degeneratively altered intervertebral disks or vertebral bodies.

### BACKGROUND OF THE INVENTION

Spinal column implants can be inserted in an intervertebral space in order to replace a removed intervertebral disk and thus to support two directly adjacent vertebral bodies against one another, either by a rigid connection or by an articulated connection. In such a case, the spinal column implants are in contact as flat implants with their contact surfaces on the front sides of the adjacent vertebral bodies.

However, spinal column implants of this type are also needed as vertebral body replacement implants, which are to bridge over one or more missing vertebral bodies. Such implants have a considerable height, because they must be at least as high as one vertebral body, and such vertebral body replacement implants thus differ markedly from intervertebral implants, which are pushed in as an intervertebral disk replacement between two vertebral bodies that are located naturally directly next to one another.

In order to guarantee the reliable support of the spinal column implant at the adjacent vertebral bodies, it is favorable in case of both vertebral body replacement implants and intervertebral implants to use the largest possible contact surfaces in order for the compressive forces to be distributed over the largest surface possible and in order to avoid pressure peaks. The contact surfaces thus frequently correspond to the area of the vertebral body surfaces, and it may therefore be difficult to introduce these spinal column implants into the body. Accesses with a large diameter are necessary for this. This prevents minimally invasive access and also makes it difficult to pass through the implant between bone parts of the skeleton, for example, between costal arches.

The same problem arises in case of implants that are not to replace a completely missing vertebral body, but are to strengthen a weakened or partially missing vertebral body, for example, when this vertebral body shows fractures because of osteoporosis. Such implants can be introduced in such cases into the vertebral body laterally through an opening prepared in the vertebral body and then pass through this vertebral body to be strengthened, and the contact surfaces of the implant come into contact with the contact surfaces of the vertebral bodies as in the case of a vertebral body replacement implant. Consequently, the implant is a vertebral body support implant in this case, whose length must bridge the entire distance between the vertebral bodies, which adjoin the weakened vertebral body that is to be strengthened on both sides, just as in a vertebral body replacement implant. Therefore, the common term spinal column implant will hereinafter be used for all implants of this type, even if the implant is an implant in the particular case that passes through a still existing vertebral body and supports it as a result.

Accordingly, there remains a need for an improved spinal column implant with at least one contact surface for support at a vertebral body that can also be introduced into the body through accesses with a smaller diameter without problems.

### SUMMARY OF THE INVENTION

The present invention comprises a spinal column implant having at least one contact surface variable in its shape or arrangement such that its cross-sectional area in a contact plane at the vertebral body is larger in a contact position than in an insertion position.

Consequently, it is ensured that even though the contact surface has the full extension in the contact position in which it is in contact with the vertebral body end face and in which it is finally implanted and thus it guarantees a good pressure distribution, it is achieved by changing the shape or the arrangement of this contact surface that the extension of the contact surface is smaller in its plane for the introduction of the implant into the body than in the final contact position. As a result, the entire implant has a smaller dimension, and this facilitates the introduction into the body through an access with a smaller diameter.

This change in the shape or arrangement of the contact surface is performed in at least one of these contact surfaces, but preferably in both contact surfaces, so that, on the whole, an implant that has smaller dimensions in the insertion state than in the final implantation state can be created for the insertion.

The shape or arrangement can be changed in a variety of ways; for example, provisions are made in a preferred embodiment for the contact surface to comprise a plurality of parts, which are brought together more closely in the insertion position to reduce the cross-sectional area than in the contact position.

In a first preferred embodiment, the parts are designed such that they can be pivoted in relation to one another and are pivoted apart from one another in the contact position in the contact plane and are pivoted toward one another in the insertion position. The pivot axis of the parts may be located in the contact plane, i.e., the parts are folded against one another by the pivoting movement.

Provisions are made in a preferred embodiment for the parts to have projections that mesh with one another in a finger-like manner and at least some of which are pivotably connected with one another at their free ends. This leads to a pivoted mounting, on the one hand, and to a largely clearance-free guiding of the pivotable parts, on the other hand.

The parts may form a right angle or an acute angle with one another in the insertion position, but it is also possible that they are pivotable against one another to the extent that they are located in parallel planes in the insertion position, and these parallel planes may extend in parallel to the direction of adjustment in an implant with contact surfaces adjustable in relation to one another at spaced locations.

It is favorable if a locking device is provided, which fixes the parts in a relative position in relation to one another. This may be the contact position, but also the insertion position, so that it is ensured that the two parts will not pivot apart from one another during the insertion.

It is favorable if the locking device is a clamping device, which clamps the parts against one another in the fixed state; in particular, the clamping device may be a locking screw.

Provisions are made in another embodiment for the locking device to comprise guide rods, which are pivotably articulated to the parts of the contact surface and are fixed at an adjusting means. These guide rods hold the parts of the contact surface in certain positions.

In particular, the guide rods may be fixed at the adjusting means displaceably, so that the contact surfaces are pivoted against one another by displacing the guide rods at the adjusting means.

For example, the guide rods may be connected with the adjusting means via a threaded connection, and pivoting of the parts of the contact surface is thus also obtained by screwing the threaded connection in or out.

Provisions are made in another preferred embodiment that the pivot axis of the parts is at right angles to the contact plane. The parts may fully or partially overlap one another, e.g., in the insertion position, and be arranged next to one another in the contact position; this can also be brought about with the use of only two parts or also with the use of a plurality of parts, which are pivoted apart or together in a fan-like pattern in this case.

It is also possible to provide a locking device that fixes the parts in an at least relative position in relation to one another; in particular, this locking device may be designed as a clamping means.

Provisions are made in another preferred embodiment for the locking device to have locking elements at the parts, which mesh with one another in a positive-locking manner in the contact position of the parts. These locking elements may be designed as projections and setbacks.

The parts may be displaceable in relation to one another, in the direction of their pivot axis, so that they are located one on top of another in the insertion position, whereas they are located next to one another in the contact position.

Provisions are made in another preferred embodiment for the parts to be able to be moved in relation to one another by means of a guide such that they partially or fully overlap in the insertion position and are arranged next to one another in the contact position. For example, the parts may be displaceable in parallel to themselves during the movement.

Provisions are made in a first preferred embodiment for the guide to have guide rods pivotably articulated to the parts.

In another embodiment, the parts are guided in relation to one another by means of parallel projections, which mesh with one another in a finger-like pattern and mesh with one another more deeply in the insertion position than in the contact position. Consequently, the parts are simply pushed together more or less in the plane in order to change the cross-sectional area.

Provisions may be made in another embodiment for the contact surface to be mounted such that it can be pivoted as a whole in relation to a bearing body around a pivot axis into a position in which the contact surface is essentially at right angles to its position in the contact position. Consequently, the contact surface as a whole is pivoted out of the contact plane in this case rather than the parts being pivoted in relation to one another, so that a smaller transverse extension is obtained in the direction of adjustment of the adjusting means and consequently in the direction in which the implant is pushed into the body.

The contact surface may be mounted transversely displaceably in relation to its pivot axis, so that it is possible to arrange the contact surface centrally over the adjusting means, but to displace it in the insertion position around a pivot axis, which is arranged at the edge at the contact surface, so that the contact surface is now arranged above the adjusting means. The pivot axis may be defined by a bearing shaft in a first preferred embodiment.

It is also possible to define the pivot axis by an arc-shaped curved path and by a bearing element guided therein.

It is favorable in such arrangements as well if a locking device is provided, which fixes the contact surface in at least one relative position in relation to a bearing body. The locking device is preferably designed as a clamping means.

According to a preferred embodiment, an adjusting means in the form of a fluid-actuated piston and cylinder unit may be provided in a spinal column implant, which is designed as a vertebral body replacement implant, to change the distance between two contact surfaces, so that a very fine adjustment can be performed by an external pressurizing agent source.

The adjusting means preferably has a fixing means for fixing its piston in relation to its cylinder in different positions, so that this distance can be fixed permanently by the fixing means after the desired distance of the contact surfaces has been reached.

In addition, the pressure in the piston and cylinder unit can now be relieved, and it is advantageous for this purpose if the adjusting means has a pressure relief valve.

Provisions are made in a preferred embodiment for at least one of the contact surfaces to be connected with the adjusting means by a detachable connection, especially an elastic locking or snap-in connection. It is possible as a result to intraoperatively equip a certain adjusting means with different contact surfaces, for example, with contact surfaces that are adapted to the transverse dimensions of the vertebral bodies to be supported or with contact surfaces that have a wedge-shaped design and thus make possible certain slopes of the supported vertebral bodies.

It is advantageous if a fixing means fixing the connection between the contact surface and the adjusting means is provided. It is ensured as a result that the contact surface is held reliably in the connection. The fixing means may be, for example, a clamping means.

It is especially advantageous if this clamping means also fixes at the same time the contact surface in a certain position in relation to the adjusting means, consequently, if the fixing means for fixing the contact surface at the adjusting means is at the same time also the locking means for fixing the position and the orientation of the contact surface in relation to the adjusting means.

According to a preferred embodiment, part of the spinal column implant may be designed as a contact plate, at which at least one support arm is mounted in such a way that it can be pivoted out. It is advantageous in this connection if the contact plate covers the support arm in the pivoted-in state. The pivot axis of the support arm may be arranged here at a longitudinal edge of the contact plate, especially in the corner area.

It is advantageous if a support arm each is mounted at opposite side edges of the contact plate.

In the pivoted-out position, the support arm can be fixed in that position by a locking mechanism and secured against pivoting in as a result; for example, the locking mechanism may be a leaf spring, which bends out from the support arm during the pivoting out.

It is advantageous if the contact plate has a depression receiving the support arm in the pivoted-in position.

Provisions may be made in another embodiment for designing a part of the implant, at which at least one support body is mounted displaceably as a contact plate. This support body may have a U-shaped design and be displaceable in parallel to its legs. It is advantageous in this case as well if the support body is fixed in the pushed-out position by a locking mechanism and is secured against being pushed in as a result.

The contact plate may have a depression receiving the support body in the pushed-in position.

Especially in embodiments with support arms that can be pivoted out or with a support body that can be pushed out, the length of the contact plate may be approximately twice its width, so that especially favorable introduction into the body is possible.

On its side facing away from the vertebral body, the contact plate may be pivotably supported at another contact plate via a joint; this is especially favorable in case of implants that are used as intervertebral implants.

The joint comprises here, according to a preferred embodiment, cooperating crowned bearing surfaces made of ceramic.

### BRIEF DESCRIPTION OF THE DRAWINGS

FIG. 1 shows a perspective view of a first preferred exemplary embodiment of a vertebral body replacement implant with contact surfaces, which are formed from two partial surfaces pivotable in relation to one another;

FIG. 2 shows a side view of the implant according to FIG. 1 in the inserted state;

FIG. 3 shows an enlarged side view of the implant according to FIG. 1;

FIG. 4 shows a top view of the implant according to FIG. 3;

FIG. 5 shows a schematic view of the implant according to FIGS. 1 through 4 with the contact surfaces in the folded-up insertion position;

FIG. 6 shows a side view of another preferred exemplary embodiment of a two-part contact surface with the parts located next to one another;

FIG. 7 shows a view similar to that in FIG. 6 with the parts pivoted one over the other;

FIG. 8 shows a top view of the implant according to FIG. 7;

FIG. 9 shows another preferred exemplary embodiment of a contact surface with two parts of a contact surface that are arranged next to one another;

FIG. 10 shows a view similar to that in FIG. 9 with parts of the contact surface pivoted one over another;

FIG. 11 shows a top view of the implant according to FIG. 10;

FIG. 12 shows another preferred exemplary embodiment of a contact surface displaced longitudinally in relation to a pivot axis in the contact position;

FIG. 13 shows a view similar to that in FIG. 12 with the contact surface in the insertion position;

FIG. 14 shows a top view of the implant according to FIG. 12;

FIG. 15 shows another preferred exemplary embodiment of a contact surface with two partial surfaces, which are mounted pivotably in relation to one another, are arranged next to one another, and are held by means of guide rods;

FIG. 16 shows a view similar to that in FIG. 15 with the partial surfaces folded up;

FIG. 17 shows a top view of another preferred exemplary embodiment of a contact surface with transversely displaceable partial surfaces in the insertion position;

FIG. 18 shows a view similar to that in FIG. 17 in the contact position;

FIG. 19 shows a top view of the implant according to FIG. 18;

FIG. 20 shows another preferred exemplary embodiment of a contact surface with an arc-shaped displacing guide in the insertion position;

FIG. 21 shows a side view of the implant according to FIG. 20;

FIG. 22 shows a view similar to that in FIG. 20 with the contact surface in the contact position;

FIGS. 23a to 23e show a side view of an implanted vertebral body support implant during the changing of the distance of the contact surfaces and the resulting unfolding of the contact surface in the contact position;

FIG. 24 shows a perspective view of a contact plate of an intervertebral implant with the support arms pivoted in;

FIG. 25 shows a side view of an intervertebral implant with two contact plates;

FIG. 26 shows a sectional view along line 26-26 in FIG. 25;

FIG. 27 shows a top view of the contact plate according to FIG. 24 with a pivot arm pivoted in and with a pivot arm pivoted out;

FIG. 28 shows a view similar to that in FIG. 27 with another preferred exemplary embodiment of a contact plate with a support arm;

FIG. 29 shows a side view of a modified exemplary embodiment of a contact plate of an intervertebral implant with an extractable support element; and

FIG. 30 shows a top view of the contact plate according to FIG. 29 with the support element in different positions.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

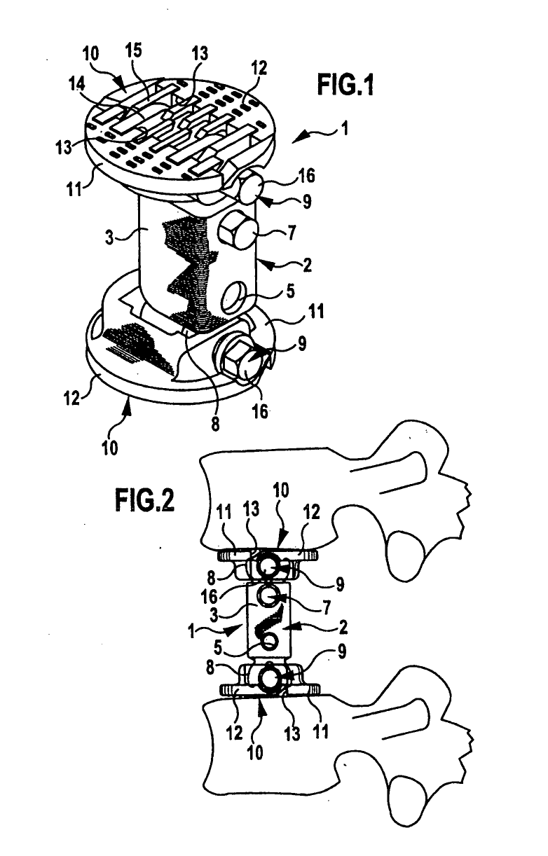

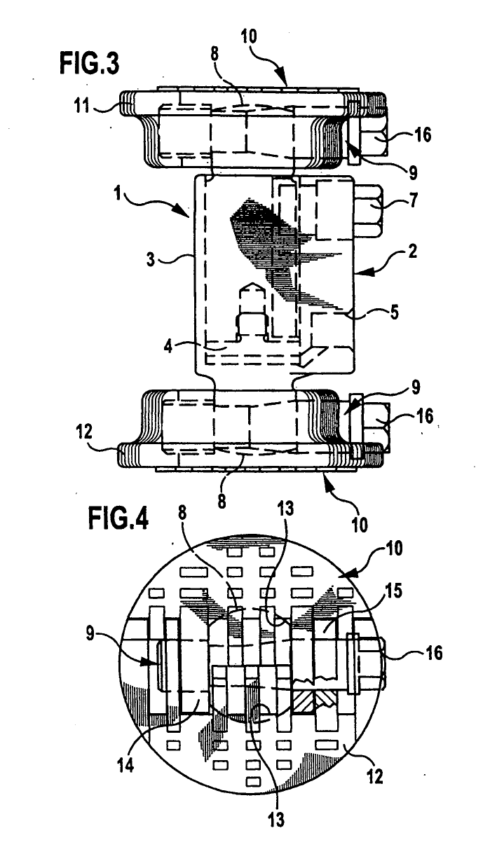

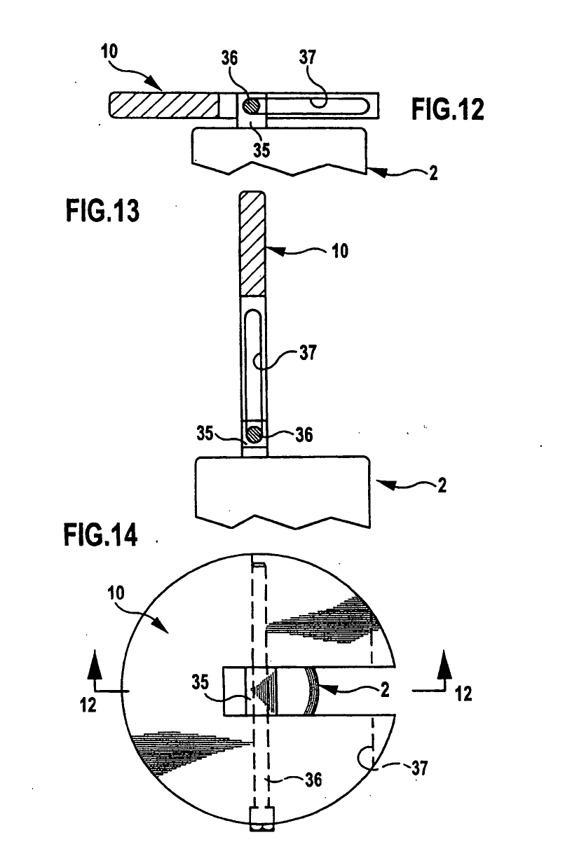

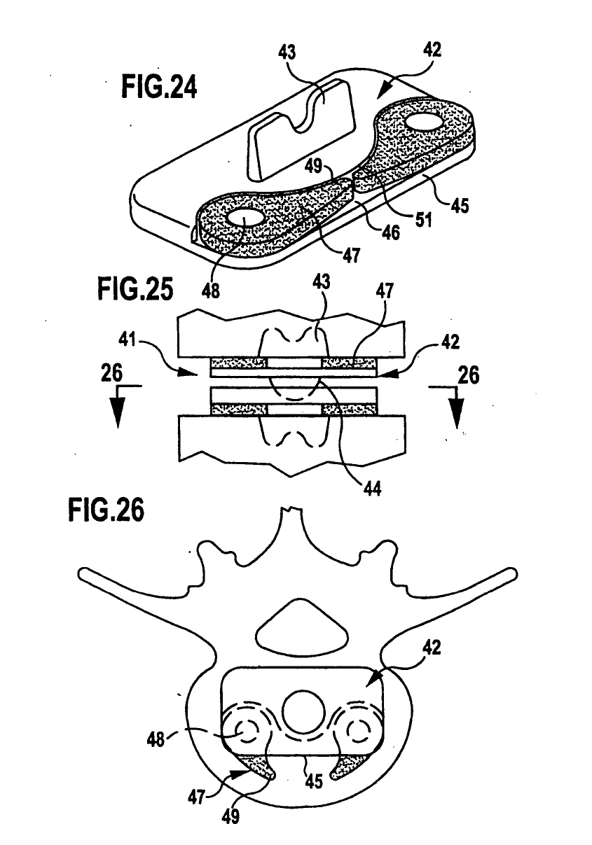

The implants shown in FIGS. 1 through 23 are vertebral body replacement implants and those in FIGS. 24 through 30 are intervertebral implants.

The implant 1 shown in FIGS. 1 through 5 comprises a piston and cylinder unit 2 with a cylinder 3 and a piston 4 mounted displaceably therein. The interior space of the piston and cylinder unit 2 can be connected via a connection opening 5 with a flexible tube 6, and this flexible tube 6 is in connection with an external reserve of a pressurizing medium (not shown), for example, a syringe-like instrument, with which a hydraulic medium, e.g., a saline solution or even a compressed gas, can be introduced into the cylinder 3, so that the piston 4 is pushed as a result out of the cylinder 3. The piston 4 can be fixed in any desired position in relation to the cylinder 3 by means of a locking screw 7 at the cylinder 3.

In addition, the interior space of the cylinder 3 can be emptied via a standard relief valve, which is not shown in the drawings, so that the interior space can be depressurized.

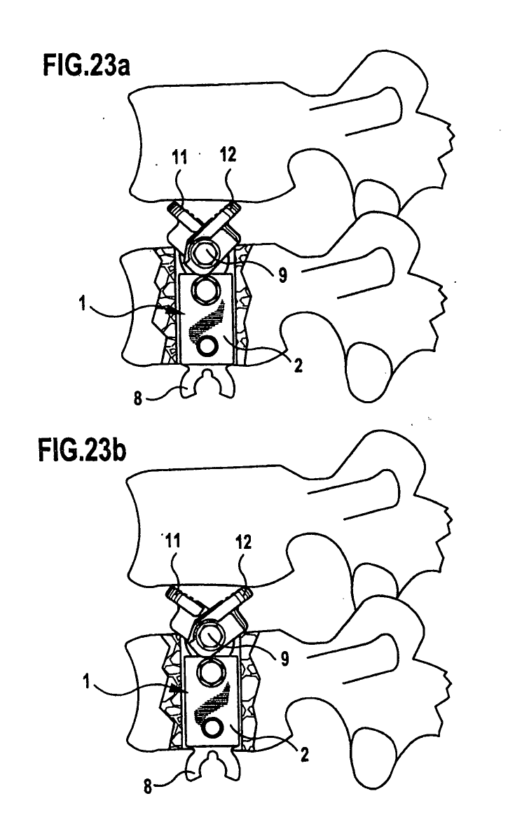

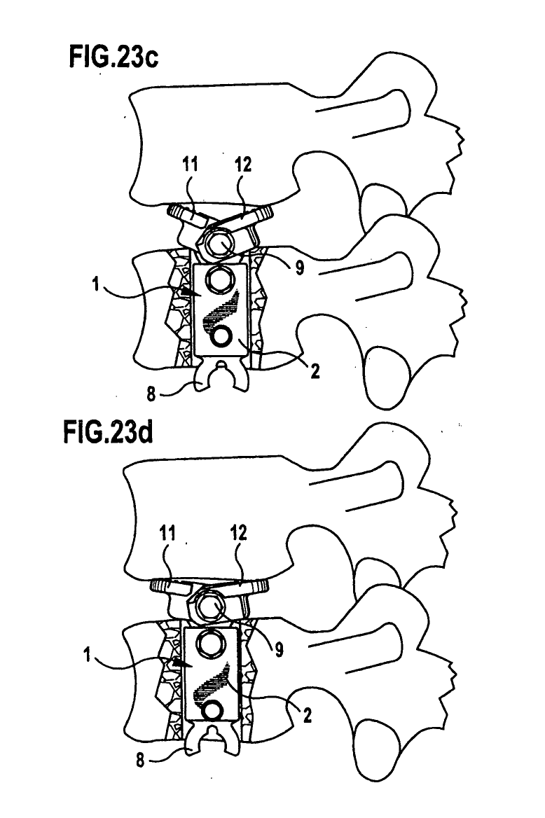

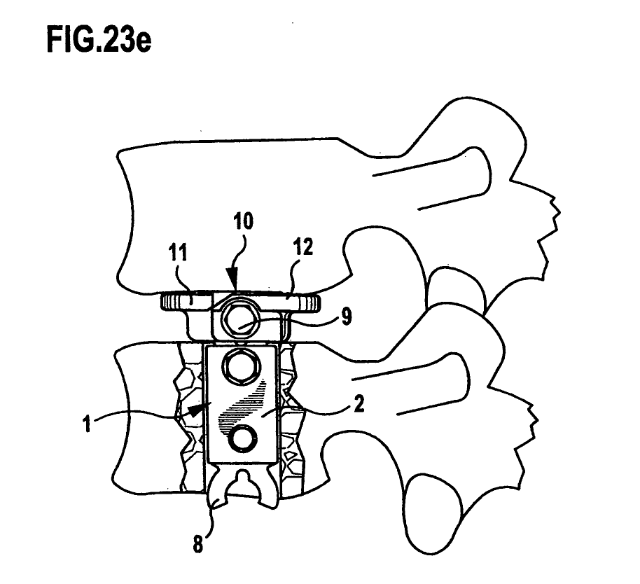

Elastically expandable, substantially U-shaped holding tongs 8 each are arranged at both the cylinder 3 and the piston 4, the holding tongs 8 having the same design and opening toward the side facing away from the piston and cylinder unit 2. Such holding tongs 8 can be clearly recognized in FIGS. 23a through 23e on the underside of the implant.

A bearing shaft 9, at which a plate-like contact surface 10 is held, can be snapped elastically into the holding tongs 8. The same design is selected on both sides of the piston and cylinder unit 2, i.e., at the cylinder 3 and at the piston 4, and the design and the function of only one of these contact surfaces will be explained in greater detail below. The contact surface 10 is composed of two parts 11, 12, which have a substantially semicircular cross section. At their inner end edges 13, both parts 11, 12 carry projections 14, 15, which extend in parallel to one another and mesh with one another in a finger-like manner, and the bearing shaft 9 is passed through at least some of these projections 14, 15 such that the two parts 11, 12 are mounted at the bearing shaft 9 pivotably around the bearing shaft 9 in relation to one another. The finger-like projections 14, 15 slide along one another during this pivoting movement and thus guide the two parts 11, 12 in the axial direction. The two parts 11, 12 can be pivoted apart completely and are located in one plane in this case.

This position, which will hereinafter be called the contact position, is defined by suitable stops. Consequently, the two parts 11, 12 form a flat contact surface 10 that is circular as a whole in this position.

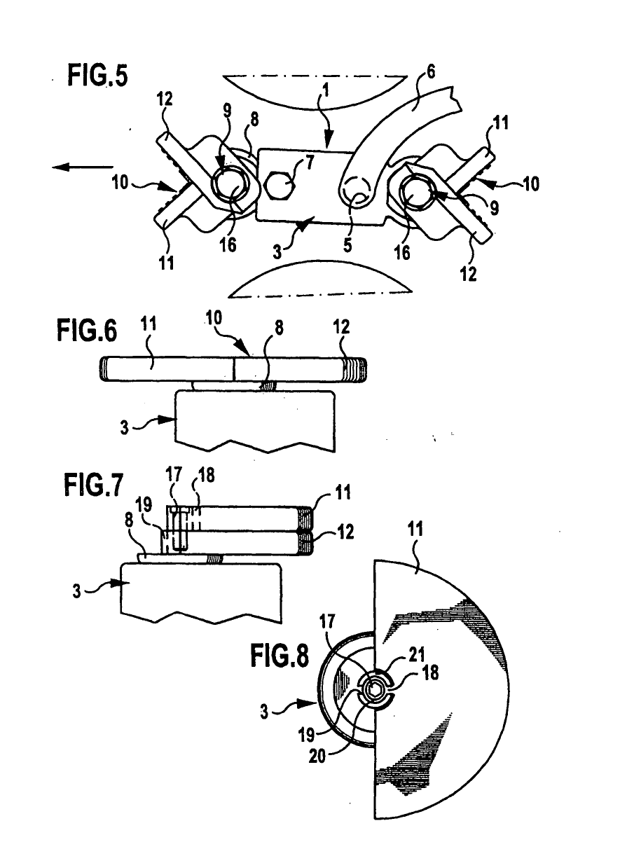

The two parts 11, 12 may be pivoted in relation to one another, and this always happens in the direction facing away from the piston and cylinder unit 2. They now form an angle between them, which is approximately a right angle in the exemplary embodiment shown in FIG. 5, but which may also be an acute angle, and this end position is defined by suitable stops. The extension of the contact surface 10 in a plane that extends at right angles to the direction of adjustment of the piston and cylinder unit 2 is smaller in this pivoted-together state, which will hereinafter be called the insertion position, than the extension of the contact surface 10 in the contact position.

The bearing shaft 9 is designed as a locking screw and is screwed for this purpose into an internal threaded section of one of the projections 14. Thus, when the bearing shaft 9 is being screwed in, it clamps together the projections 14 and 15 of the two parts 11, 12 of the contact surface 10 and fixes same as a result in its corresponding angular position. The bearing shaft 9 carries a hexagon head 16 for this purpose, to which a screwing-in tool can be attached.

This hexagon head 16 is located directly above the locking screw 7, so that both the bearing shaft 9 and the locking screw 7 can be actuated from the same side.

The bearing shaft 9 is also fixed by this clamping in the holding tongs 8, because the projections 14, 15 are also pressed against the lateral surfaces of the holding tongs 8, and the assembly unit comprising the two parts 11, 12 of the contact surface 10, on the one hand, and of the bearing shaft 9, on the other hand, are thus securely fixed in the holding tongs 8 when the bearing shaft 9 is screwed in.

The assembly unit comprising the contact surface 10 and the bearing shaft 9 may be easily replaced at the piston and cylinder unit 2. The bearing shaft 9 is loosened, after which the bearing shaft 9 can be extracted from the holding tongs 8 and another assembly unit can be inserted. It is thus possible to select intraoperatively the assembly unit comprising the contact surface and the bearing shaft that is particularly needed for the special purpose of the surgery, and rapid replacement is also possible if necessary.

To introduce the implant 1 into the body, the two parts 11, 12 are pivoted against one another into the insertion position and then fixed by tightening the bearing shaft 9. The extension of the implant 1 at right angles to the direction of adjustment of the piston and cylinder unit 2 is thus relatively small, so that the piston and cylinder unit 2 can be introduced into the body through accesses with a small diameter without problems (FIG. 5).

After the introduction, the contact surface 10 can be unfolded inside the body after loosening the bearing shaft 9 into the contact position, in which the two parts 11, 12 are located in one plane, and the implant 1 can then be pushed into the intermediate place between the remaining adjacent vertebral bodies in place of a missing vertebral body.

The procedure illustrated in FIGS. 23a through 23e can be followed in the case of an implant that is not to replace a missing vertebral body, but only strengthen a weakened vertebral body. An opening is prepared laterally in the weakened vertebral body, and the implant 1 is pushed through this opening into the vertebral body after it has been introduced into the body. For clarity purposes, a contact surface 10 is shown in FIGS. 23a through 23e only at the upper end of the piston and cylinder unit 2, and not at the lower end. However, it is contemplated that contact surfaces of the same type are also used at the lower end. The view is shown without a lower contact surface to show the holding tongs 8 clearly.

The implant 1 may be pushed into the vertebral body to be strengthened in the insertion position, so that the edges of the parts 11, 12 of the contact surface 10 will first come into contact with the vertebral bodies to be supported (FIG. 23a). By distracting the piston and cylinder unit 2, the contact surfaces are successively pressed against the vertebral bodies to be supported, and the vertebral body end faces unfold the two parts 11, 12 in the process until these are finally located in one plane (FIGS. 23b through 23e). The bearing shaft 9 can be clamped in this position, and the two parts 11, 12 are thus fixed in their pivoted-out contact position; in addition, the contact surface 10 and the bearing shaft 9 are fixed in the holding tongs 8.

This operation is carried out in the same manner at both ends of the implant 1, and the distance reached by the piston and cylinder unit 2 can be fixed after this operation by tightening the locking screw 7, and the piston and cylinder unit 2 can be subsequently relieved, i.e., the pressurizing medium is removed from the piston and cylinder unit 2, and the distance once reached between the contact surfaces 10 is maintained because of the action of the locking screw 7.

The same procedure may, of course, also be followed in case of an implant that is used to replace a missing vertebral body.

While the two parts 11, 12 of the contact surface 10 in the exemplary embodiment according to FIGS. 1 through 5 as well as 23a through 23e are pivotable in relation to one another around an axis that extends at right angles to the direction of adjustment of the piston and cylinder unit 2, FIGS. 6 through 8 show an exemplary embodiment in which the two parts 11, 12 are rotatable in relation to one another around an axis that extends in parallel to the direction of adjustment. A similar design is otherwise selected, and parts that correspond to one another therefore carry the same reference numbers.

The two parts 11, 12 are semicircular in this case and have no projections meshing with one another in a finger-like manner, but they are in contact with one another with their end edges 13 in the contact position and thus form a continuous circular contact surface 10. One of the two parts is rigidly connected with the cylinder 3 and the piston 4, respectively, and the other of the two parts is mounted, in contrast, rotatably in relation to the first part. A bearing bolt 17, which passes through one part 11 and is screwed into the other part 12 and also acts as a locking screw in the clamped state at the same time and thus fixes the rotatable part 11, is used for mounting. This rotatable part 11 can be displaced in the direction of the bearing bolt 17 such that it is located next to the part 12 in the contact position (FIG. 6), whereas it is located on the part 12, covering the same, in the insertion position (FIG. 7).

While the mutual fixation can be performed by the action of the bearing bolt 17 acting as a locking screw, an additional or exclusive fixation may also be achieved by positive locking. For example, the pivotable part 11 may immerse with a web 18 into a groove 19 of the stationary part 12 when the part 11 is in the contact position (FIG. 8). The web 18 now connects the part 11 with a bearing eye 20 through which the bearing bolt 17 passes, and the groove 19 is located in a collar 21 of the stationary part 12, which collar 21 surrounds the bearing bolt 17.

The overall width of the contact surface is reduced in the insertion position by the two parts 11 and 12 covering one another.

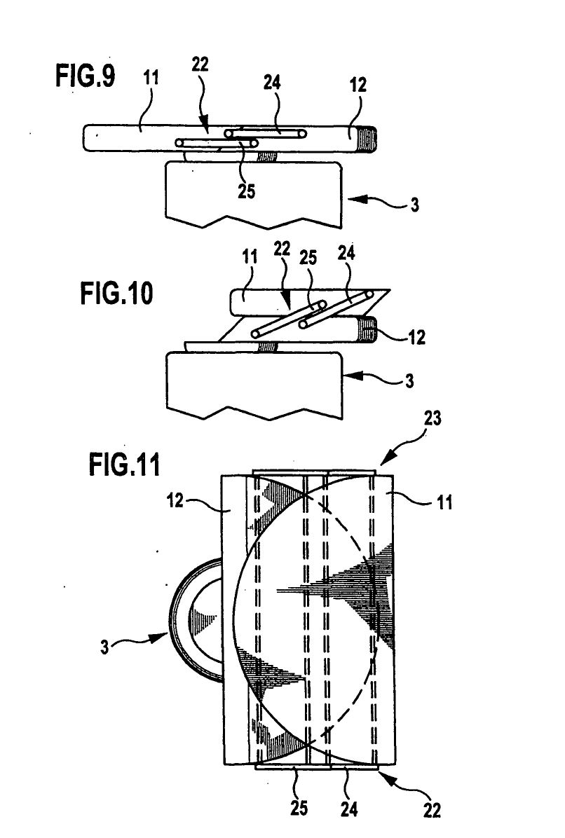

A similar embodiment is shown in the exemplary embodiment according to FIGS. 9 through 11, parts corresponding to one another being designated by the same reference numbers. The two parts 11, 12 are connected to one another in this case via guide rod pairs 22, 23 arranged on opposite sides, and each guide rod pair is formed by two parallel guide rods 24, 25, and part 11 is thus mounted displaceably in parallel to itself at the part 12 rigidly connected to the piston and cylinder unit 2 via a parallelogram guide. The displacement may take place between a contact position in which the part 11 is arranged next to the part 12 in the same plane as this (FIG. 9), and an insertion position in which the part 11 is arranged on the part 12, covering the same (FIGS. 10 and 11).

As in the exemplary embodiment according to FIGS. 6 and 8, the extension of the contact surface is larger in the contact position than in the insertion position in this exemplary embodiment as well.

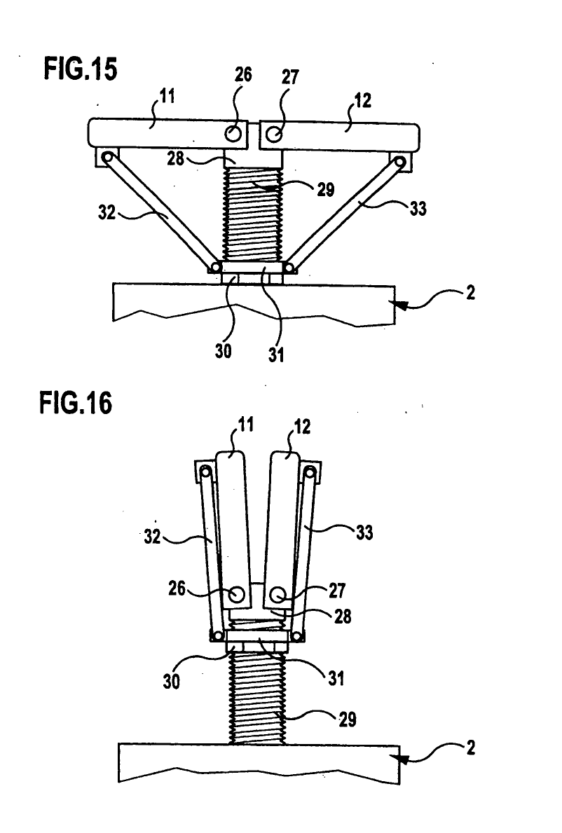

In the exemplary embodiment according to FIGS. 15 and 16, in which a design similar to that in the exemplary embodiment according to FIGS. 1 through 5 is selected, and in which parts that correspond to one another have the same reference numbers, the two parts 11, 12 are connected pivotably around a respective pivot axis 26 and 27 of their own with the bearing post 28, which itself carries an external thread 29. A nut 30 is screwed onto the external thread 29, the nut 30 being moved during the screwing together along the external thread 29 and is rotatably and axially nondisplaceably connected with a retaining ring 31, at which a guide rod 32, 33, is each mounted pivotably on opposite sides. These guide rods 32 and 33 are pivotably connected with the parts 11, 12, so that the parts 11, 12 can be pivoted during the displacement of the nut 30 along the external thread 29 from a lower position, in which they extend in parallel to one another in the same plane and thus define the contact position (FIG. 15), into an upper position, in which they are folded up and, extending essentially in parallel to each other, project upwardly (FIG. 16). The extension of the parts 11, 12 in a plane extending at right angles to the direction of displacement of the piston and cylinder unit 2 is considerably smaller in the folded-up state than in the contact position, in which the two parts 11, 12 are located in a common plane.

The nut 30 acts as a locking mechanism at the same time, and provisions may additionally be made for the pivot axes 26, 27 to be formed by locking screws, which fix the angular position of the parts 11, 12 at the bearing post 28, similar to the bearing shaft 9.

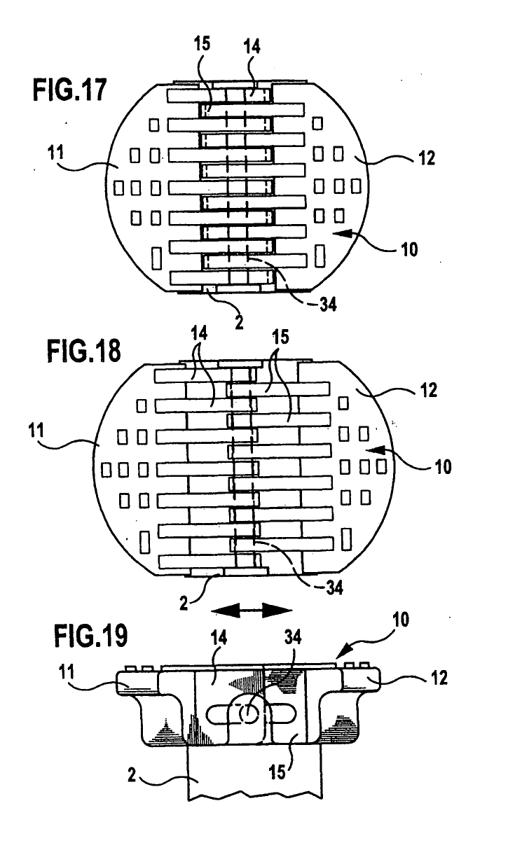

At least one of the two parts 11, 12, which otherwise have a design similar to that in the exemplary embodiment according to FIGS. 1 through 5, is displaceable in the plane of the contact surface 10 in relation to the other part in the exemplary embodiment according to FIGS. 17 through 19, and this displacing movement is guided by projections 14, 15, which mesh with one another in a finger-like manner and mesh with one another more deeply with the parts 11, 12 pushed together than in the case in which the parts 11, 12 are pulled apart. The insertion position is assumed in the pushed-together state (FIG. 17), and the contact position in the pulled-apart state (FIG. 18). The transverse extension of the contact surface 10 is markedly smaller in the insertion position than in the contact position.

The relative positions of the two parts 11, 12 can be fixed in this case as well, for example, by a locking screw 34, which passes through both parts 11, 12 and is shown only schematically in the views in FIGS. 17 through 19.

While the contact surface comprises a plurality of parts that can be pivoted or displaced in relation to one another in the devices explained thus far, the exemplary embodiment according to FIGS. 12 through 14 shows a one-part contact surface 10. The design selected is otherwise similar to that in the exemplary embodiment according to FIGS. 1 through 5.

The one-part contact surface 10 is mounted pivotably at a bearing projection 35 of the cylinder 3 and of the piston 4 by means of a hinge pin 36, and this hinge pin 36 engages an elongated hole guide 37 in the contact surface 10, so that the contact surface 10 can be displaced in relation to the hinge pin 36.

In the contact position, the hinge pin 36 is at one end of the elongated hole guide 37 and approximately in the middle of the contact surface 10. In contrast, the contact surface 10 is first displaced in the insertion position on the hinge pin 36 to the extent that the hinge pin 36 strikes the other end of the elongated hole guide 37, i.e., in the area of the contact surface 10 near the edge. The entire contact surface 10 can be pivoted upward by 90° in this position, so that it will thus be directed upward in the extension of the piston and cylinder unit 2 (FIG. 14). The extension of the contact surface 10 at right angles to the direction of displacement of the piston and cylinder unit 2 is markedly smaller in this insertion position than in the contact position according to FIG. 12. The hinge pin 36 may be designed as a locking screw in this case as well, and it can fix the contact surface 10 in any desired angular position in relation to the bearing projection 35.

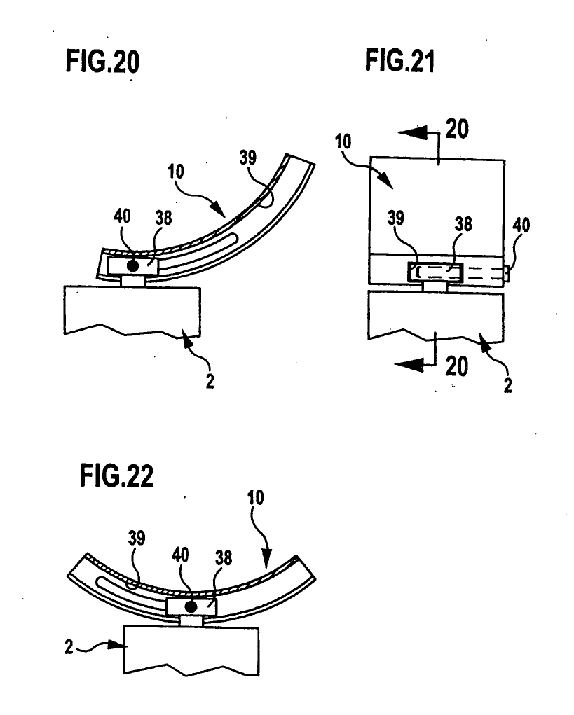

A displacing movement of the contact surface 10 in relation to the piston and cylinder unit 2 can be achieved not only by means of bearing shafts, but also by the suitable guiding of guide elements in guideways. In the exemplary embodiment according to FIGS. 20 through 22, the piston and cylinder unit 2 carries such a guide element 38 in the form of an expanding projection, which meshes in a positive-locking manner with an arc-shaped guideway 39 of a contact surface 10, only a lower part of which is shown in the views in FIGS. 20 through 22. It becomes clear from the schematic views in FIGS. 20 through 22 that tilting of the contact surface 10 out of the contact position, in which this embodiment extends at right angles to the direction of displacement of the piston and cylinder unit 2, into a tilted position, in which the contact surface 10 is pivoted at least partially or completely in the direction of the displacement and has a smaller width at right angles thereto as a result, is possible in this way as well.

The guide element 38 can be fixed by a locking screw 40 in relation to the guideway 39 in any desired position in this embodiment.

Provisions may be made in all the exemplary embodiments described for the contact surface 10 to be held alone or together with its bearing elements detachably and replaceably at the piston and cylinder unit 2, as this was described, for example, in the exemplary embodiment according to FIGS. 1 through 5 in respect to the snapping in of the bearing shaft 9 into the holding tongs 8. It is thus always possible to intraoperatively connect different contact surfaces with the piston and cylinder unit 2 and thus meet the particular requirements in terms of the geometry of the contact surfaces. These may also have an extension that is smaller than the extension of the supported vertebral end faces, as this becomes clear, for example, from the exemplary embodiment shown in FIGS. 23a through 23e.

While the above exemplary embodiments represent vertebral body replacement implants, which are inserted to bridge over a vertebral body defect, FIGS. 24 through 30 show intervertebral implants, which can be inserted into the intervertebral space between two adjacent vertebral bodies after the removal of the intervertebral disk.

The intervertebral implant 41 shown in FIGS. 24 through 27 comprises an approximately rectangular, oblong contact surface 42, which is about twice as long as it is wide. The length corresponds here approximately to the transverse dimension of the vertebral body support surface, whereas the contact surface 42 at right angles thereto is only about half the width of the vertebral body contact surface. A substantially rectangular shape is selected in the exemplary embodiment shown. However, it is also contemplated that a kidney shape may be selected, or a shape bent in any other way, which is adapted to the contour of the vertebral body surface.

On one side, the contact plate 42 carries anchoring projections 43, which penetrate the vertebral body in contact with the anchoring projections 43 and fix the contact plate 42.

Two such contact plates 42 together form an intervertebral implant 41, and two contact plates 42 are provided for this purpose with crowned, mutually complementary joint surfaces 44, which are two-dimensionally in contact with one another and are formed, for example, by ceramic inlay bodies, which are firmly inserted into corresponding recesses of the contact plate 42. As a result, the two contact plates 42 pivotably support one another and can be pivoted in relation to one another within certain limits.

Each of the two contact plates 42 has, along a longitudinal edge 45, a depression 46, which receive two support arms 47 each. The two support arms 47 have a mirror symmetrical design in the exemplary embodiment shown in FIGS. 24 through 27, and only one of the support arms 47 will therefore be explained in greater detail. The support arm 47 is mounted pivotably at the contact plate 42 around a pivot axis 48 extending perpendicularly on the contact plate 42, and the pivot axis 48 is located in a corner area. In this area, the support arm 47 surrounds the pivot axis 48 in the manner of an eye and extends with an extension part 49 approximately up to the middle of the contact plate 42 when both support arms 47 are pivoted into the depression 46 (FIG. 24). In this position, the extension parts 49 are located in the middle of the contact plate 42 directly opposite one another, and the support arms 47 are completely covered by the contact plate 42.

Both support arms 47 can be pivoted out of this position, so that the extension part 49 projects beyond the outer contour of the contact plate 42 and enlarges the effective contact surface of the contact plate 42 as a result (FIG. 26). A leaf spring 50, which performs an excursion during the pivoting out of the support arm 47 and is in contact by its free end with an edge 51 of the depression 46 such that the support arm 47 cannot be pivoted back into the pivoted-in position any longer, is inserted laterally into the support arm 47. Securing against the unintended pivoting in of the support arm 47 is thus achieved.

Only the two support arms 47 are replaced with a single support arm 47, which extends essentially over the entire length of the contact plate 42, in the exemplary embodiment according to FIG. 28, which has essentially the same design and in which identical parts are therefore designated by the same reference numbers. The extension part 49 has an arc-shaped design, so that the effective contact surface is increased as much as possible during the pivoting out of the support arm 47 and the extension part 49 extends over the edge area of the vertebral body, which has an especially high strength. This also applies to the support arms 47 in the exemplary embodiment according to FIGS. 24 through 27, in which the extension parts 49 also extend into the especially stable edge area of the vertebral body and therefore support the contact plate especially effectively at the vertebral body.

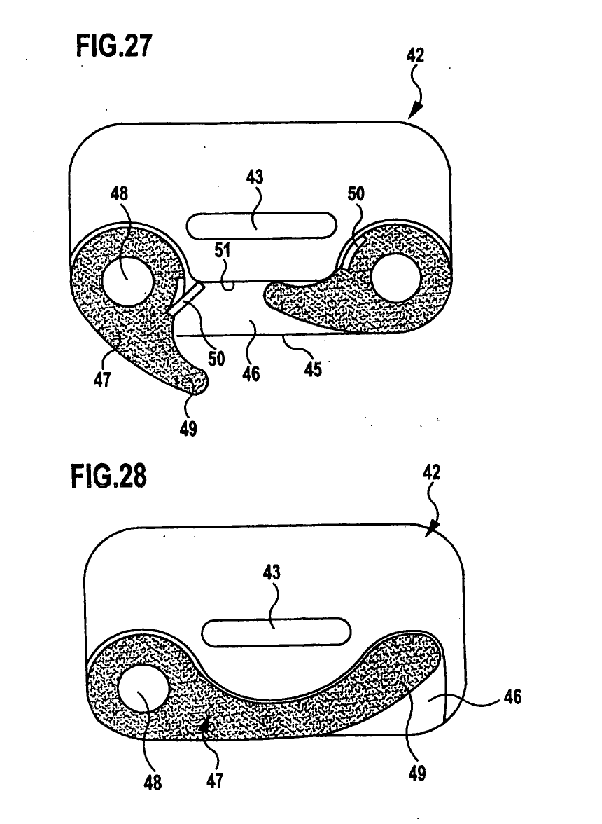

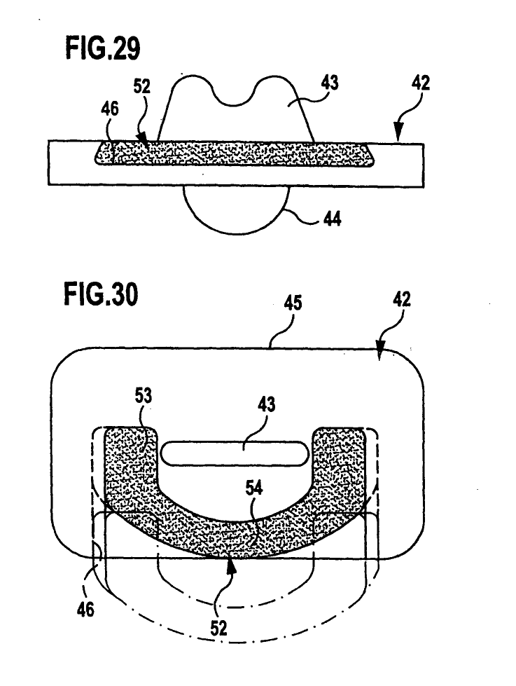

While pivotable support arms are used in the exemplary embodiments according to FIGS. 24 through 28 to enlarge the effective contact surface of the contact plate 42, the contact plate 42 of the exemplary embodiment according to FIGS. 29 and 30, which otherwise has a similar design and in which identical parts are designated by the same reference numbers, has a support element 52, which has a U-shaped design and thus has two parallel legs 53 and a bent web 54 connecting these legs 53. This support element 52 is mounted displaceably in parallel to its legs 53 in the depression 46 and can thus be displaced from a pushed-in position, in which the contact plate 42 completely covers the support element 52 (indicated by solid lines in FIG. 30) into a pushed-out position (indicated by dash-dotted lines in FIG. 30), in which the web 54 and parts of the legs 53 project beyond the contour of the contact plate 42 and thus enlarge the effective contact surface of the contact plate 42. Locking may also be provided in this case, for example, with the use of a leaf spring, as described above with reference to FIG. 27.

Due to the relatively small dimensions of the contact plate 42, it is possible to introduce this intervertebral implant 41 into the intervertebral space from the side rather than ventrally, as is otherwise common, and implantation can be performed as a result, even in cases in which ventral introduction would cause difficulties or would be impossible because of the anatomic conditions. Nevertheless, the effective contact surface of the contact plate 42 can be enlarged by pivoting out or extracting the support arms 47 or support elements 52 to the extent that the supporting forces are distributed over a very large contact surface, so that there is no risk of the contact plates 42 breaking into the vertebral bodies.

The various designs to enlarge the contact surfaces may be interchanged between vertebral replacement implants and intervertebral implants as desired, i.e., the designs described in the examples on the basis of intervertebral implants and vertebral body replacement implants are not limited to these alone.

Biocompatible metals, especially titanium alloys or chromium-cobalt alloys, are preferably used as the material for all of the above-described parts 10, 11, 12, and 42. As an alternative, components may be made of plastic, especially from PEEK™, which is a polymer (polyether ether ketone) manufactured by Victrex® PLC of the United Kingdom. PEEK™ is transparent to X-rays, which leads to a great advantage in postoperative X-ray diagnostics with CTs or nuclear spin tomography, because, unlike metals, the plastic does not cause any artifacts (i.e., obstructions) in the X-ray image.

To minimize wear, components may be made of ceramic. Such ceramic components are manufactured with corresponding precision such that the wear nearly equals zero. A further advantage of a ceramic-on-ceramic bearing is that the problem of creep under load, which is peculiar to polyethylene, is absent. Since ceramic material has a substantially higher compressive strength and dimensional stability than polyethylene, dimensions may be reduced. The forced translational motion superimposed to the flexion/extension movement decreases as a result.

The components described above may be mounted substantially without clearance, because abrasion may otherwise occur at ceramic/metal interfaces because of the hardness of the ceramic material. This clearance-free mounting/assembly can be achieved, e.g., by means of a conical clamping. However, other possibilities of the clearance-free mounting/assembly can be exhausted as well, such as: shrinking of the parts onto bearing surfaces by means of thermal expansion; use of elastic intermediate elements (not shown), which compensate a clearance between components due to their intrinsic elasticity/deformation; and additional locking screws (not shown).

The intervertebral disk prosthesis can be inserted with the aid of navigated instruments. In use, components are assembled prior to implantation, and the intervertebral disk prosthesis is implanted in the assembled state, thereby significantly simplifying the implantation procedure.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

1. A spinal column implant for contact with a vertebral body, said implant comprising:
at least one contact surface comprising a contact plane having a cross-sectional area in contact with the vertebral body,
wherein the shape and arrangement of said at least one contact surface is adjustable such that said cross-sectional area of said contact plane is larger in a contact position than in an insertion position.

2. The implant of claim 1, wherein said contact surface comprises a plurality of moveable parts which are adjusted more closely in relation to one another, to reduce said cross-sectional area of said contact plane, in said insertion position than in said contact position.

3. The implant of claim 2, wherein said parts are displaceable in relation to one another along a pivot axis, are pivoted apart in said contact position, and are pivoted toward one another in said insertion position.

4. The implant of claim 3, wherein said pivot axis of extends in the contact plane.

5. The implant of claim 4, wherein said parts comprise projections which mesh with one another in a finger-like manner and at least some of which are pivotably connected to one another at their free ends.

6. The implant of claim 4, wherein said parts form a right or acute angle with respect to one another in said insertion position.

7. The implant of claim 4, wherein said parts are positioned along parallel planes in said insertion position.

8. The implant of claims 4 further comprising a locking device for fixing said parts in a relative position in relation to one another.

9. The implant of claim 8, wherein said locking device is a clamping means for clamping said parts in a fixed state in relation to one another.

10. The implant of claim 9, wherein said clamping means is a locking screw.

11. The implant of claim 8, wherein said locking device comprises guide rods pivotably articulated to said parts of said contact surface and fixed at an adjusting means.

12. The implant of claim 11, wherein said guide rods are displaceably fixed at said adjusting means.

13. The implant of claim 12, wherein said guide rods are connected with said adjusting means via a threaded connection.

14. The implant of claim 3, wherein said pivot axis of said parts extends perpendicularly from said contact plane.

15. The implant of claim 14, wherein said parts fully or partially cover one another in said insertion position and are arranged adjacent one another in said contact position.

16. The implant of claim 14 further comprising a locking device for fixing said parts in a relative position in relation to one another.

17. The implant of claim 16, wherein said locking device is a clamping means.

18. The implant of claim 16, wherein said locking device comprises locking elements located on said parts, and said locking elements mesh with one another in a positive-locking manner in said contact position of said parts.

19. The implant of claim 18, wherein said locking elements (18, 19) are designed as a projection and a cutout.

20. The implant of claim 14, wherein said parts are displaceable in relation to one another in the direction of said pivot axis.

21. The implant of claim 2 further comprising a guide, wherein said parts can be moved in relation to one another by means of a said guide such that they partially or fully cover one another in said insertion position and are arranged adjacent one another in said contact position.

22. The implant of claim 21, wherein said parts are displaceable in parallel to themselves during movement.

23. The implant of claim 21, wherein said guide comprises guide rods articulated pivotably to said parts.

24. The implant of claim 21 further comprising parallel projections, wherein said parts are guided in relation to one another by means of said parallel projections that mesh with one another in a finger-like manner and mesh with one another more deeply in said insertion position than in said contact position.

25. The implant of claim 3 further comprising a bearing body, wherein said contact surface is mounted in such a way that it can be pivoted as a whole in relation to said bearing body around said pivot axis into a position in which said contact surface is substantially perpendicular to the position it assumes in said contact position.

26. The implant of claim 25, wherein said contact surface is mounted transversely displaceably in relation to said pivot axis.

27. The implant of claim 25, wherein said pivot axis is defined by a bearing shaft.

28. The implant of claim 25 further comprising a bearing element, wherein said pivot axis is defined by an arc-shaped curved path and said bearing element guided therein.

29. The implant of claim 25 further comprising a locking device for fixing said contact surface in at least one relative position in relation to an adjusting means.

30. The implant of claim 29, wherein said locking device is a clamping means.

31. The implant of claim 11, wherein said adjusting means comprises a fluid-actuated piston and cylinder unit, said unit adapted to change the distance between said contact surfaces.

32. The implant of claim 31, wherein said adjusting means further comprises a fixing means for fixing said piston in relation to said cylinder in different positions.

33. The implant of claim 31, wherein said adjusting means further comprises a pressure relief valve.

34. The implant of claim 11, wherein said at least one contact surface is connected with said adjusting means by a detachable connection.

35. The implant of claim 34, wherein said detachable connection is an elastic locking or snap-in connection.

36. The implant of claim 34 further comprising a fixing means for fixing said detachable connection of said contact surface with said adjusting means.

37. The implant of claim 36, wherein said fixing means is a clamping means.

38. The implant of claim 37, wherein said clamping means fixes said contact surface in a certain position simultaneously in relation to said adjusting means.

39. The implant of claim 14 further comprising at least one support arm, wherein one part is designed as said contact plate on which said at least one support arm is pivotally mounted on a pivot axis such that it can be pivoted toward a pivoted-out position.

40. The implant of claim 39, wherein said contact plate covers said support arm in a pivoted-in position.

41. The implant of claim 39, wherein said pivot axis of said support arm extends at a longitudinal edge of said contact plate.

42. The implant of claim 39, wherein a support arm is mounted at opposite side edges of said contact plate.

43. The implant of claim 39, wherein said support arm is secured in said pivoted-out position via a locking mechanism, and is thereby secured against pivoting in.

44. The implant of claim 43, wherein said locking mechanism is a leaf spring bending away from said support arm when said support arm is pivoted out.

45. The implant of 40, wherein said contact plate comprises a depression for receiving said support arm in said pivoted-in position.

46. The implant of claim 22, wherein one part is designed as said contact plate on which at least one support body is displaceably mounted.

47. The implant of claim 46, support body comprising legs, wherein said support body is U-shaped and is displaceable in parallel to said legs.

48. The implant of claim 46, wherein said support body is fixed in a pushed-out position via a locking mechanism, and is thereby secured against being pushed in toward a pushed-in position.

49. The implant of claims 48, wherein said contact plate comprises a depression for receiving said support body in said pushed-in position.

50. The implant of claim 39, wherein the length of said contact plate is about twice its width.

51. The implant of claim 39, wherein said contact plate is supported in conjunction with another contact plate via a hinge on its side facing away from the vertebral body.

52. The implant of claim 51, wherein said joint comprises cooperating crowned bearing surfaces made of ceramic.
